(19) United States
(12) Patent Application Publication
(10) Pub. No.: US 2015/0037405 A1
(43) Pub. Date: Feb. 5, 2015

(54) PHARMACEUTICAL COMPOSITIONS OF LEVODOPA AND CARBIDOPA

(71) Applicant: CADILA HEALTHCARE LIMITED, Gujarat (IN)

(72) Inventors: Sushrut Krishnaji Kulkarni, Gujarat (IN); Pavak Rajnikant Mehta, Gujarat (IN); Sameer Sagar Kochar, Gujarat (IN); Krishnakumar Vinubhai Patel, Gujarat (IN)

(21) Appl. No.: 14/450,475

(22) Filed: Aug. 4, 2014

(30) Foreign Application Priority Data
Aug. 5, 2013 (IN) 2576/MUM/2013

(51) Int. Cl. A61K 31/197; A61K 47/12; A61K 9/48; A61K 9/16; A61K 9/28

(52) U.S. Cl. CPC A61K 31/197; A61K 9/1682; A61K 9/2893; A61K 9/4833; A61K 9/4808; A61K 9/2886; A61K 9/167; A61K 47/12; A61K 9/2833
USPC 424/458; 514/565; 424/457; 427/2.21

(57) ABSTRACT

The present invention relates to pharmaceutical compositions of levodopa and carbidopa. In particular, the invention relates to modified release pharmaceutical compositions of levodopa and carbidopa with at least one organic acidic excipient. The invention also relates to processes for the preparation of such compositions and use thereof for treatment of Parkinson's disease.

### FIELD OF THE INVENTION

The present invention relates to pharmaceutical compositions of levodopa and carbidopa. In particular, the invention relates to modified release pharmaceutical compositions of levodopa and carbidopa with at least one organic acidic excipient. The invention also relates to processes for the preparation of such compositions and use thereof for treatment of Parkinson's disease.

### BACKGROUND OF THE INVENTION

Combinations of levodopa (LD) and carbidopa to treat Parkinson's disease (PD) are known in the pharmaceutical art and are considered by many to be the ‘gold standard’ treatment for symptoms of PD. Currently, several compositions containing a combination of LD and CD are commercially available, e.g., SINEMET®, STALEVO®, PARCOPA®, and ATAMET®. Nonetheless, a need remains for an oral LD composition that provides steadier plasma concentrations of LD with minimal ‘peak-to-trough’ fluctuations during daily dosing and that yields a longer duration-of-effect than the currently available oral dosage forms of CD/LD.

Patients suffering from PD frequently have periods in which their mobility becomes difficult, often resulting in an inability to move. Abnormally low levels of dopamine, a neurotransmitter that affects mobility and control of the skeletal-muscular system, is commonly believed to be the cause of these motor symptoms in PD patients. However, administration of dopamine is not effective to treat Parkinson's disease because dopamine does not cross the blood-brain barrier. To resolve this problem, PD patients are administered levodopa, the metabolic precursor of dopamine, but levodopa is not without its issues either.

While levodopa crosses the blood-brain barrier and is rapidly converted to dopamine, LD is problematic because of its rapid decarboxylation by tissues other than the brain. Thus, when LD is administered alone, large doses are required because only a small portion is transported to the brain unchanged. Furthermore, when levodopa is administered orally it is rapidly decarboxylated to dopamine in extracerebral tissues so that only a small portion of a given dose is transported unchanged to the central nervous system. Carbidopa inhibits the decarboxylation of peripheral levodopa and does not cross the blood-brain barrier. Since its decarboxylase inhibiting activity is limited to extracerebral tissues, administration of carbidopa with levodopa has been popular to make levodopa more available for transport to the brain.

PD patients treated with LD may develop motor fluctuations characterized by end-of-dose failure, peak dose dyskinesia, and akinesia. The advanced form of motor fluctuations (also commonly referred to as the ‘on-off’ phenomenon) is characterized by unpredictable swings from mobility to immobility. Although the causes of these motor fluctuations are not completely understood, some patients may be attenuated by treatment regimens that produce steady plasma levels of LD. Thus, a void remains in the LD treatment of PD patients, as plasma concentration levels remain difficult to control.

Currently available controlled release compositions of CD/LD are meant to allow for a continuous release of drug over a prolonged period of time in an attempt to maintain tight LD plasma ranges. However, the use of these controlled release dosage forms are problematic in that many PD patients wake up in the morning having little or no mobility due to the wearing off of the dose taken the day/evening before. Once the previous dose has worn off, such patients are usually unwilling, or even unable, to wait for the extended period of time required for a controlled release dosage form to deliver the necessary plasma levels of LD. While the use of an immediate release composition of LD can reduce this ‘wait time’, the use of an immediate release composition of LD require more frequent dosing and are associated with more fluctuating plasma LD concentrations. DUODOPA®, an intraduodenal infusion therapy approved outside of the United States, demonstrates significantly reduced motor complications and reduced ‘off’ time. The cumulative experiences from DUODOPA® and experimental infusion studies show that the maintenance of stable plasma LD concentrations and the avoidance of low trough levels appear to be effective in reducing ‘off’ time, increasing ‘on’ time without disabling dyskinesia, and reduce the severity of dyskinesia in comparison to the standard oral compositions. However, such infusion therapies are extremely inconvenient to the patients.

The results of infusion therapies, such as DUODOPA®, strongly suggest a rationale for the development of a LD treatment that provide constant, or relatively steady, LD plasma concentrations to optimize relief of PD symptoms and to minimize ‘off’ times and dyskinesias. Indeed, a need remains for a more convenient, i.e., oral dosage form that will improve the administration of LD to PD patients by narrowing blood plasma ranges of LD, which in turn will result in reduced ‘off times’, prolonged ‘on time’, and decreased time to ‘on’.

WO 2009085306 discloses multiparticulate controlled release oral solid composition of levodopa comprising levodopa, carbidopa and carboxylic acid.

US publication no. 2007/0148238 discloses oral dosage form of levodopa and carbidopa that allows the drug to be released in highly controlled and time-dependent manner.

US publication no. 2007/0003621 discloses plurality of pellets comprising cabidopa, levodopa, wherein one portion of pellets are coated with bioadhesive polymers.

European Patent No. 0253490 discloses a composition of LD and CD uniformly dispersed in a polymeric matrix consisting of a mixture of two polymers, one of which is water-soluble, such as hydroxypropyl methylcellulose, and the other of which is weakly water soluble polymer, such as polyvinyl acetate/crotonic acid copolymer. This tablet contains a release release-controlling polymer.

U.S. Pat. No. 5,192,550 discloses an osmotic device for administering a drug, for example LD and/or CD, for treating central nervous system disorders. The device comprises a first composition comprising a pharmaceutical drug carrier and 100 nanograms to 700 milligrams of drug granules.

U.S. Pat. No. 5,532,274 and No. 5,624,960 disclose a composition having controlled liberation of LD and CD during a short release phase comprising a polymer mixture consisting of polyvinyl alcohols.

U.S. Pat. No. 6,117,453 discloses a solid pharmaceutical composition comprising an active ingredient, such as LD or CD, which is not in an amorphous form, polyethylene oxide, and the balance consisting of conventional additives, excluding basic components. This composition contains a release rate controlling polymer.

U.S. Pat. No. 6,217,905 discloses a dosage form for administering an anti-Parkinson drug to a patient, wherein the dosage form comprises: “(a) a composition comprising 0.10 mg to 750 mg of an anti-Parkinson drug and a pharmaceutically acceptable carrier for the anti-Parkinson drug selected from the group consisting of hydroxypropylcellulose, hydroxypropyl methylcellulose and polyvinylpyrrolidone, which composition in the presence of fluid that contacts the dosage form provides a dispensable anti-Parkinson therapeutic composition; and wherein the dosage form: (b) provides the anti-Parkinson drug substantially-free of adverse effects for administration in a rate-controlled metered dose per unit time over 24 hours. In one embodiment, the anti-Parkinson drug is a combination of LD and CD.

U.S. Pat. No. 6,238,699 and its related U.S. Pat. No. 6,756,056 disclose a pharmaceutical tablet comprising a sustained release core layer of CD (25-75 mg), LD (100-400 mg), cellulose ether (80 mg), and microcrystalline cellulose, wherein the sustained release layer is overcoated with an immediate release layer comprising CD (10-25 mg) and LD (50-200 mg), wherein the sustained and immediate release layers are separated by a drug free excipient layer. A bilayer tablet consisting of one layer of sustained release carbidopa-levodopa adjacent to a layer of immediate release carbidopa-levodopa is also disclosed. This tablet contains a release rate-controlling polymer.

U.S. Pat. No. 6,372,254 discloses a press-coated tablet suitable for oral administration, comprising an immediate-release compartment comprising a compressed blend of an active agent, such as LD, and one or more polymers, and an extended-release compartment, formed by press-coating to substantially envelop the immediate-release compartment, and comprising a compressed blend of the active agent, a hydrophilic polymer and hydrophobic material, wherein the tablet exhibits a first order release of the active agent interrupted by a pulsed delivery of the active agent. This tablet contains a release rate-controlling polymer.

U.S. Pat. No. 6,531,153 discloses a pharmaceutical composition comprising a therapeutically effective amount of LD and of CD, dispersed in a hydrophilic matrix, and an organic acid. The hydrophilic matrix generally comprises a gelling substance such as hydroxypropyl methylcellulose. Other gelling components may be used, such as polyvinylpyrrolidone, poly(vinyl alcohol), hydroxypropylcellulose, hydroxymethylcellulose or gelatin, alone or as a mixture. This pharmaceutical composition contains a release rate-controlling polymer.

U.S. Pat. No. 6,607,751 and U.S. Patent Publication No. 2004/0009219 disclose a controlled release pharmaceutical device that comprises pharmaceutically active substances, such as LD or CD, microbial polysaccharide, and uncrosslinked linear polymer, such as cellulose ether. When the delivery device of this invention is administered to the gastrointestinal tract by oral route it comes into contact with an aqueous environment and hydrates forming a gelatinous layer. The device provides sustained or pulsatile delivery of pharmaceutically active substances for a predetermined period of time. The duration, uniformity and continuity of release of the pharmaceutically active agent(s) can be suitably controlled by varying the relative amount of the xanthan gum and HPMC. This composition contains a release rate-controlling polymer.

The present invention provides a novel controlled release oral solid dosage form of LD that is formulated with carbidopa and an organic acidic excipient to yield the desired pharmacokinetic attributes.

### SUMMARY OF THE INVENTION

In one general aspect there is provided a pharmaceutical composition comprising levodopa, carbidopa, at least one organic acidic excipient and one or more pharmaceutically acceptable excipients.

In another general aspect there is provided a solid dosage form comprising levodopa, carbidopa, at least one organic acidic excipient and one or more pharmaceutically acceptable excipients.

In another general aspect there is provided a controlled release pharmaceutical composition comprising levodopa, carbidopa, at least one organic acidic excipient and one or more pharmaceutically acceptable excipients.

In another general aspect there is provided a pharmaceutical composition of levodopa, carbidopa, at least one organic acidic excipient, one or more disintegrating agents, release controlling excipients, diluents, binders, surfactants, lubricants, glidants, sweeteners and flavoring agents.

In another general aspect there is provided a modified release pharmaceutical composition comprising:
a controlled release component of levodopa and carbidopa,
a delayed release component of levodopa and carbidopa; and
a delayed release component of at least one organic acidic excipient,
   wherein the composition is devoid of any immediate release component of levodopa and carbidopa.

In another general aspect there is provided a stable pharmaceutical composition comprising levodopa, carbidopa, at least one organic acidic excipient and one or more pharmaceutical excipients and retains at least 80% of the potency of levodopa and carbidopa in the pharmaceutical composition after storage at 40° C. and 75% relative humidity for three months.

In another general aspect there is provided a process for preparing a pharmaceutical composition of levodopa, carbidopa, at least one organic acidic excipient. The process comprises the steps of making drug components having controlled release coating, preparing a component of organic acidic excipient with controlled release coating, encapsulating drug components and organic acidic excipient component to make a final composition.

In another general aspect there is provided a method of treating parkinson's disease in patient comprising administering to said subject a pharmaceutical composition comprising levodopa, carbidopa, at least one organic acidic excipient and one or more pharmaceutically acceptable excipients.

The details of one or more embodiments of the present invention are set forth in the description below. Other features, objects and advantages of the invention will be apparent from the description.

### DETAILED DESCRIPTION OF THE INVENTION

We have surprisingly found that modified release pharmaceutical compositions of levodopa and carbidopa can be prepared using at least one organic acidic excipient and such compositions provide steady or constant, LD plasma concentrations in patients, resulting in decreased motor-fluctuations, reduced ‘off’ time and increased ‘on’ time in PD patients.

The inventors have developed pharmaceutical compositions of levodopa and carbidopa using at least one organic acidic excipient. In particular, the inventors have developed pharmaceutical compositions by careful selection of organic acids and/or organic acid salts with their optimum concentrations.

Moreover, such compositions are also stable and may retain at least 80% of the potency of levodopa and carbidopa in the pharmaceutical composition after storage at 40° C. and 75% relative humidity for three months.

The phrase “pharmaceutically acceptable” is employed herein to refer to those compounds, materials, compositions; and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The term “component” includes, but not limited to beads, granules, pellets, tablets, minitablets, microcapsules, spheres or particles.

The term “organic acidic excipient” may include a carboxylic acid or pharmaceutically acceptable salt thereof those known to person skilled in the art. Non limiting examples of such carboxylic acids are tartaric acid, adipic acid, succinic acid, citric acid, benzoic acid, acetic acid, ascorbic acid, edetic acid, fumaric acid, lactic acid, malic acid, oleic acid, sorbic acid, stearic acid and palmitic acid or mixtures thereof. Non limiting examples of such organic acid salts are alkaline earth metal salts such as sodium, potassium, lithium, calcium, strontium, barium, and antimony; ammonium salts; and amine or alkanolamine salts of the carboxylic acid.

The term “tablet” or “pill” comprises a pharmaceutical composition pressed into a form. The form can be in any shape, for example, round, oblong, triangular or other shapes.

The term “modified release” (also known as MR) includes delayed release (also known as DR) and controlled release (also known as CR, sustained release (SR), prolonged release (PR) or extended release (ER)).

The term “immediate release” (also known as instant release or IR) refers to a pharmaceutical composition or component thereof which releases, or delivers, one or more pharmaceutical agents substantially immediately upon administration and will result in more than about 80% within about half an hour.

The term “about” when used in connection with percentages means±10% of its original value.

Embodiments of the present invention relate to pharmaceutical compositions of levodopa and carbidopa, at least one organic acidic excipient and one or more pharmaceutically acceptable excipients.

The pharmaceutical composition of the present invention refers to pharmaceutical compositions which can be formulated into powder, granule, fine granule/micro-granules, pellets, wafers, minitablets, tablet or capsule.

In one embodiment, the pharmaceutical composition comprises levodopa, carbidopa, at least one organic acidic excipient and one or more pharmaceutically acceptable excipients.

In another embodiment, the pharmaceutical composition comprises levodopa, carbidopa, at least one organic acid and one or more pharmaceutically acceptable excipients.

In another embodiment, the pharmaceutical composition comprises levodopa, carbidopa, at least one organic acid salt and one or more pharmaceutically acceptable excipients.

In another embodiment, the pharmaceutical composition comprises about 25 mg to 2000 mg of levodopa and about 10 mg to 300 mg of carbidopa and at least one organic acidic excipient.

In another embodiment, the pharmaceutical compositions of levodopa, carbidopa and at least one organic acid, optionally have functional as well non-function coating. The functional coatings may include controlled-release and/or delayed release coating and non-functional coating may include seal coatings and/or elegant coatings.

In another embodiment, the modified release pharmaceutical composition comprises levodopa, carbidopa, at least one organic acidic excipient and a release controlling excipient.

In another embodiment, the modified release oral solid dosage form comprises levodopa, carbidopa, at least one organic acidic excipient and a release controlling excipient, wherein the modified release solid dosage form is a matrix tablet or a capsule composition.

In another embodiment, the modified release pharmaceutical composition comprises
a controlled release component of levodopa and carbidopa,
a delayed release component of levodopa and carbidopa; and
a delayed release component of at least one organic acidic excipient.

In another embodiment, the modified release pharmaceutical composition comprises
controlled release minitablets of levodopa and carbidopa,
delayed release minitablets of levodopa and carbidopa; and
delayed release minitablets of at least one organic acidic excipient.

In another embodiment, the modified release pharmaceutical composition comprises
controlled release pellets of levodopa and carbidopa,
delayed release minitablets of levodopa and carbidopa; and
delayed release minitablets of at least one organic acidic excipient.

In another embodiment, the modified release pharmaceutical composition comprises
controlled release minitablets of levodopa and carbidopa,
delayed release pellets of levodopa and carbidopa; and
delayed release minitablets of at least one organic acidic excipient.

In another embodiment, the modified release pharmaceutical composition comprises
controlled release pellets of levodopa and carbidopa,
delayed release pellets of levodopa and carbidopa; and
delayed release tablets/minitablets of at least one organic acidic excipient.

In another embodiment, the modified release pharmaceutical composition of the present invention provides an in-vivo plasma profile for carbidopa as mentioned below, when administered in a fasted state:

a mean of Cₘₐₓ more than about 40 ng/mL,
a mean of AUC_{0-∞} more than about 150 ng*hr/mL; or
a mean of Tₘₐₓ at least about 3 hours.

In another embodiment, the modified release pharmaceutical composition of the present invention provides an in-vivo plasma profile for levodopa as mentioned below, when administered in a fasted state:

a mean of Cₘₐₓ more than about 600 ng/mL,
a mean of AUC_{0-∞} more than about 2000 ng*hr/mL; or
a mean of Tₘₐₓ at least about 3 hours.

Suitable “release controlling excipients” include pH dependent and pH independent release polymers.

The pH dependent release polymers are the excipients whose performance is dependent on the pH of the medium. A number of such excipients known in the art include poly methacrylic acid derivatives, cellulose derivatives, acrylic acid derivatives, maleic acid copolymers, polyvinyl derivatives etc. Cellulose based pH dependent release polymers include hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose acetate trimelliate, cellulose benzoate phthalate, cellulose propionate phthalate, methylcellulose phthalate, carboxymethylethylcellulose, ethylhydroxyethylcellulose phthalate and the like. Acrylic copolymer based pH dependent release polymers include styrene/acrylic acid copolymer, methyl acrylate/acrylic acid copolymer, methyl acrylate/methacrylic acid copolymer, butyl acrylate styrene/acrylic acid copolymer, methacrylic acid/methyl methacrylate copolymer (e.g. Trade-names: Eudragit L 100 and Eudragit S 100, available from Röhm Pharma), methacrylic acid ethyl acrylate copolymer (e.g. Trade-name: Eudragit L 100-55, available from Röhm Pharma), methyl acrylate/methacrylic acid/octyl acrylate copolymer. Maleic copolymer based pH dependent release polymers include vinylacetate/maleic acid anhydride copolymer, styrene maleic acid anhydride copolymer, styrene maleic acid monoester copolymer, vinylnethylether-maleic acid anhydride copolymer, ethylene-maleic acid anhydride copolymer, vinylbutylether maleic acid anhydride copolymer, acrylonitrile methyl acrylate maleic acid anhydride copolymer, butyl acrylate styrene maleic acid anhydride copolymer and the like. Polyvinyl derivative based pH dependent release polymers include polyvinyl alcohol phthalate, polyvinylacetal phthalate, polyvinyl butylate phthalate, polyvinylacetoacetal phthalate and the like.

The pH independent release polymers are the excipients whose performance is independent of the pH of the environment. The pH independent release polymers include, but not limited to polyvinyl alcohol, polyvinyl acetate, Kollidon SR (which is a mixture of 8 parts w/w of polyvinyl acetate and 2 parts w/w of polyvinylpyrrolidone), Polymethacrylic acid derivatives, cellulose derivatives such as ethyl cellulose, hydroxypropylmethylcellulose, triglycerides, waxes such as compritol, gelucires, lipids, fatty acids or their salts or derivatives such as stearic acid, etc.

In another embodiment, the pharmaceutical composition may be provided as a multiparticulate composition, which may be encapsulated or pressed into a tablet.

Alternatively, the multiparticulates may be in a sprinkle form that can be sprinkled directly onto food or liquids for easy ingestion.

In another embodiment, the pharmaceutical composition may comprise two distinct components of active ingredients, wherein one component may be coated with one or more pH independent release polymers, while the remaining component may be coated with one or more pH dependent release polymers.

In another embodiment, the pharmaceutical composition comprises levodopa, carbidopa and at least one organic acidic excipient, wherein the desired dissolution profile of the pharmaceutical composition according to the present invention can be achieved by using organic acidic excipientls in the range of about 5 to 40% by weight of the total weight of composition.

The pharmaceutically acceptable excipients for use in the pharmaceutical composition of levodopa and carbidopa may include one or more diluents/fillers/bulking agents, binders, disintegrants, lubricants, glidants, sweeteners/taste masking agents, colorants and flavors.

Suitable diluents/fillers or bulking agents include, but are not limited to, microcrystalline cellulose, di- or tri-basic calcium phosphate, crystalline cellulose, powdered cellulose, calcium carbonate, calcium sulphate, magnesium silicate, magnesium trisilicate, magnesium aluminium metasilicate (Neusilin), kaolin, starch, starch derivatives, magnesium carbonate, magnesium oxide and co-processed insoluble excipients.

Suitable binders include, but are not limited to, hydroxyethyl cellulose, hydroxypropyl cellulose, hydroxypropyl methylcellulose, carbomers, dextrin, ethyl cellulose, methylcellulose, shellac, zein, gelatin, gum arabic, polymethacrylates, polyvinyl pyrrolidone, polyvinyl alcohol, polyethylene glycol, carrageenan, polyethylene oxide, waxes, pullulan, agar, tragacanth, veegum, pregelatinized starch, sodium alginate, gums, sugars such as sucrose, maltose, dextrose, lactose, amylase, synthetic resins and the like.

Suitable disintegrants include, but are not limited to, Veegum (highly refined isomorphous silicate), crospovidone, cellulose, kaolin, crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof. Preferred disintegrants among these disintegrants include crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof. The amount of disintegrant in the pharmaceutical composition ranges from about 0.5% to about 10% by total weight of the composition.

Suitable lubricants and glidants include, but are not limited to, stearic acid and its derivatives or esters like sodium stearate, magnesium stearate and calcium stearate and the corresponding esters such as sodium stearyl fumarate; talc and colloidal silicon dioxide.

Suitable taste masking agents include, but are not limited to, one or more of polymers, surfactants, sweeteners and flavors. Examples of polymers include one or more of cellulose acetate, polymethacrylates, cellulose derivatives such as hydroxypropylmethyl cellulose, hydroxypropyl cellulose, hydroxylethyl cellulose; and the like. Examples of sweeteners include but not limiting to one or more of aspartame, saccharin, sucralose, glycyrrhizin; and the like.

Suitable sweeteners include, but are not limited to, saccharides such as aspartame, sugar derivatives. Other examples of sweeteners comprise sodium saccharin; aspartame; sugarless sweeteners, hydrogenated starch hydrolysates, alone or in combination.

Suitable flavors include, but are not limited to, cinnamon, wintergreen, eucalyptus, spearmint, peppermint, menthol, anise as well as fruit flavors such as apple, pear, peach, vanilla, strawberry, cherry, apricot, orange, watermelon, banana and the like; bean-derived flavors, such as coffee, cocoa and the like or mixtures thereof.

In another embodiment, the modified release pharmaceutical composition of levodopa and carbidopa may be prepared by
preparing drug pellets of carbidopa and levodopa,
coating one part of drug pellets with one or more pH independent polymers,
coating the remaining part of drug pellets with one or more pH dependent polymers,
preparing core tablets of organic acidic excipient,
coating the tablets of organic acidic excipient with controlled release polymers; and
encapsulating coated drug pellets and coated tablets of organic acidic excipient.

The pharmaceutical compositions of the present invention may be prepared by the methods known to the person skilled in the art. Pellets may be prepared by extrusion spheronization, solution and suspension layering, spray drying or spray congealing techniques. Tablets may be prepared by dry granulation, wet granulation, direct compression or melt granulation.

The invention further provides a method of treating Parkinson's disease in patient comprising administering to said subject a pharmaceutical composition comprises levodopa, carbidopa, at least one organic acidic excipient.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| | | |
|---|---|---|
| | | Quantity |
| Sr. No. | Ingredients | (mg) |

Preparing pellets of levodopa and carbidopa
with controlled release coating (Component A)

| | | |
|---|---|---|
| 1. | Carbidopa | 24.50 |
| 2. | Levodopa | 98.00 |
| 3. | Microcrystalline cellulose | 28.65 |
| 4. | Mannitol | 8.92 |
| 5. | Sodium starch glycolate | 4.37 |
| 6. | Sodium lauryl sulfate | 8.75 |
| 7. | Povidone | 1.75 |
| 8. | Butylated hydroxyl anisole | 0.06 |
| 9. | Ethanol | q.s. |
| 10. | Water | q.s. |

Composition of controlled release coating

| | | |
|---|---|---|
| 11. | Ethylcellulose | 7.87 |
| 12. | Hypromellose | 5.25 |
| 13. | Dichloromethane | q.s. |
| 14. | Methyl alcohol | q.s. |

| | |
|---|---|
| Total of component A | 188.12 |

Preparing pellets of levodopa and carbidopa
with delayed release coating (Component B)

| | | |
|---|---|---|
| 1. | Carbidopa | 36.75 |
| 2. | Levodopa | 147.00 |
| 3. | Microcrystalline cellulose | 63.51 |
| 4. | Colloidal silicone dioxide | 2.55 |
| 5. | Hypromellose | 5.10 |
| 6. | Butylated hydroxy anisole | 0.09 |
| 7. | Ethanol | q.s |
| 8. | Water | q.s. |

Seal coating composition

| | | |
|---|---|---|
| 9. | Hypromellose | 12.75 |
| 10. | Dichloromethane | q.s. |
| 11. | Methyl alcohol | q.s. |

Composition of delayed release coating

| | | |
|---|---|---|
| 12. | Eudragit L100 | 3.30 |
| 13. | Eudragit S100 | 6.57 |
| 14. | Triethyl citrate | 2.81 |
| 15. | Talc | 2.84 |
| 16. | Acetone | q.s. |
| 17. | Isopropyl alcohol | q.s. |
| 18. | Purified water | q.s. |

| | |
|---|---|
| Total of component B | 283.28 |

Organic acidic excipient with
delayed release coating (Component C)

| | | |
|---|---|---|
| 1. | Tartaric acid | 133 |
| 2. | Microcrystalline cellulose | 39.12 |
| 3. | Hypromellose | 45.00 |
| 4. | Colloidal silicone dioxide | 2.25 |
| 5. | Magnesium stearate | 5.62 |

Composition of controlled release coating

| | | |
|---|---|---|
| 6. | Ethylcellulose | 11.47 |
| 7. | Hypromellose | 3.82 |
| 8. | Triethyl citrate | 2.7 |
| 9. | Dichloromethane | q.s. |
| 10. | Methyl alcohol | q.s. |

Composition of delayed release coating

| | | |
|---|---|---|
| 11. | Eudragit L100 | 15.4 |
| 12. | Triethyl citrate | 4.39 |
| 13. | Talc | 4.44 |
| 14. | Acetone | q.s. |
| 15. | Isopropyl alcohol | q.s. |
| 16. | Purified water | q.s. |

| | |
|---|---|
| Total of component C | 267.30 |

### Procedure:

### Preparation of Component A

Screened carbidopa, levodopa, microcrystalline cellulose, mannitol, sodium starch glycolate, sodium lauryl sulfate and povidone mixed in rapid mixer granulator. This blend was granulated with butylated hydroxyl anisole in ethanol followed by purified water. This wet mass was extruded in an extruder using suitable screen. Extrudes were charged and spheronized in a spheronizer equipped with suitable cross-hatch disc. The spheres obtained were dried and screened through suitable mesh screen. These drug pellets were coated with solution of ethylcellulose and hydroxypropyl methylcellulose in dichloromethane and methyl alcohol.

### Preparation of Component B

Screened carbidopa, levodopa, microcrystalline cellulose and colloidal silicone dioxide were mixed in rapid mixer granulator. This blend was granulated with butylated hydroxyl anisole in ethanol followed by solution of hypromellose in purified water. This wet mass was extruded in an extruder using suitable screen. Extrudes were charged and spheronized in a spheronizer equipped with suitable cross-hatch disc. The spheres obtained were dried and screened through suitable mesh screen. These drug pellets were seal coated with solution of hydroxypropyl methylcellulose in dichloromethane and methyl alcohol. These seal coated drug pellets were further coated with Eudragit L100, Eudragit S100, triethyl citrate, talc in mixture of isopropyl alcohol, acetone and purified water using wurster column assembly.

### Preparation of Component C

Screened tartaric acid, microcrystalline cellulose, hypromellose and colloidal silicone dioxide were loaded in suitable blender and mixed to get uniform blend. This blend was lubricated with prescreened magnesium stearate. Lubricated blend of tartaric acid was compressed into tablets using round shape punches using suitable dies. These core tablets were coated with solution of ethylcellulose, hydroxypropyl methylcellulose and triethyl citrate in dichloromethane and methyl alcohol. These controlled release coated tablets were further coated with dispersion of Eudragit L100, triethyl citrate and talc in mixture of isopropyl alcohol, acetone and purified water using suitable tablet coating assembly.

Required parts of component A, component B and component C were filled in a capsule to make a final composition.

### Dissolution Test:

Individual components obtained by above method and the composite capsules were subjected to the dissolution test according to United States Pharmacopoeia, Dissolution apparatus 2, under following conditions:

Dissolution Test of Component a (Pellets of Levodopa and Carbidopa with Controlled Release Coating)
Dissolution Medium: 0.1 N HCl
Paddle rotation: 50 rpm
Volume of the medium: 900 ml

The results of dissolution test are shown below:

| | Dissolution (% of drug dissolved) | |
|---|---|---|
| Time (Hours) | Carbidopa | Levodopa |
| 0.5 | 66.6 | 67.0 |
| 1 | 90.1 | 93.2 |
| 1.5 | 95.7 | 100.8 |
| 2 | 96.8 | 102.1 |

Dissolution Test of Component B (Pellets of Levodopa and Carbidopa with Delayed Release Coating)

### Conditions for First Two Hours:

Dissolution Medium: 0.1 N HCl
Paddle rotation: 50 rpm
Volume of the medium: 900 ml
   Conditions from Third to Seventh Hour:
Dissolution Medium: pH 6.8 phosphate buffer
Paddle rotation: 50 rpm
Volume of the medium: 900 ml

The results of dissolution test are shown below:

| | Dissolution (% of drug dissolved) | |
|---|---|---|
| Time (Hours) | Carbidopa | Levodopa |

(900 ml 0.1N HCl, paddle, 50 rpm)

| | | |
|---|---|---|
| 0.25 | 0.0 | 0.0 |
| 1 | 0.0 | 0.0 |
| 2 | 0.0 | 0.0 |

(900 ml pH 6.8 phosphate buffer, paddle, 50 rpm)

| | | |
|---|---|---|
| 3 | 29.5 | 26.7 |
| 4 | 56.8 | 55.1 |
| 5 | 70.4 | 73.0 |
| 6 | 77.8 | 85.1 |
| 7 | 78.7 | 93.1 |

### Dissolution Test of Composite Capsules:

### Conditions for First 30 min:

Dissolution Medium: 0.1 N HCl
Paddle rotation: 50 rpm
Volume of the medium: 900 ml
   Conditions from 30 min to Eighth Hour:
Dissolution Medium: pH 6.8 phosphate buffer
Paddle rotation: 50 rpm
Volume of the medium: 900 ml

The results of dissolution test are shown below:

| | Dissolution (% of drug dissolved) | |
|---|---|---|
| Time (Hr) | Carbidopa | Levodopa |

(900 ml 0.1N HCl, paddle, 50 rpm)

| | | |
|---|---|---|
| 0.25 | 11.6 | 11.2 |
| 0.5 | 23.3 | 23.2 |

(900 ml pH 6.8 phosphate buffer, paddle, 50 rpm)

| | | |
|---|---|---|
| 1 | 40.1 | 38.6 |
| 2 | 62.6 | 61.2 |
| 3 | 71.8 | 75.5 |
| 4 | 75.9 | 83.1 |
| 5 | 76.1 | 89.7 |
| 6 | 76.6 | 93.4 |
| 8 | 74.7 | 96.3 |

### Pharmacokinetic Results:

Pharmacokinetics tests were carried out in 15 subjects in fasted state. The results are shown below:

| Pharmacokinetic | | |
|---|---|---|
| parameters | Carbidopa | Levodopa |
| Mean Cₘₐₓ (ng/ml) | 44.71 | 674.74 |
| Mean AUC_{0-∞} (ng · hr/ml) | 183.30 | 2469.1 |
| Mean Tₘₐₓ (hr) | 4.07 | 3.78 |

1. A modified release pharmaceutical composition comprising
a controlled release component of levodopa and carbidopa,
a delayed release component of levodopa and carbidopa; and
a delayed release component of at least one organic acidic excipient,
wherein the composition is devoid of any immediate release component of levodopa and carbidopa.

2. The modified release pharmaceutical composition according claim 1, wherein the organic acidic excipient is an organic acid or an organic acid salt.

3. The modified release pharmaceutical composition according to claim 1, wherein the organic acidic excipient is an organic acid comprising one or more of tartaric acid, adipic acid, succinic acid, citric acid, benzoic acid, acetic acid, ascorbic acid, edetic acid, fumaric acid, lactic acid, malic acid, oleic acid, sorbic acid, stearic acid or palmitic acid.

4. The modified release pharmaceutical composition according to claim 1, wherein the organic acidic excipient is an organic acid salt comprising one or more of sodium, potassium, lithium, calcium, strontium, barium, antimony; ammonium salts; or amine or alkanolamine salts of a carboxylic acid.

5. The modified release pharmaceutical composition according to claim 1 comprising about 25 mg to 2000 mg of levodopa and about 10 mg to 300 mg of carbidopa.

6. The modified release pharmaceutical composition according to claim 1, wherein the composition retains at least 80% of the potency of levodopa and carbidopa in the said composition after storage for three months at 40° C. and 75% relative humidity.

7. The modified release pharmaceutical composition according to claim 1, wherein the controlled release component and the delayed release components are present in the form of pellets, granules, tablets, minitablets or combinations thereof.

8. The modified release pharmaceutical composition according to claim 1, wherein the organic acidic excipient is present in an amount of 5 to 40% w/w of the total composition.

9. The modified release pharmaceutical composition according to claim 1, wherein the composition is in the form of a multiparticulate composition.

10. The modified release pharmaceutical composition according to claim 1, wherein the multiparticulates are filled into a capsule.

11. The modified release pharmaceutical composition according to claim 1, wherein the multiparticulates are pressed into a tablet.

12. The modified release pharmaceutical composition according to claim 1, wherein the controlled release or the delayed release component comprising one or more pH independent polymers, pH dependent polymers or combinations thereof.

13. The modified release pharmaceutical composition according to claim 12, wherein pH independent polymers comprising one or more of polymethacrylic acid derivatives, cellulose derivatives, acrylic acid derivatives, maleic acid copolymers or polyvinyl derivatives.

14. The modified release pharmaceutical composition according to claim 12, wherein pH, independent polymers comprising one or more of polyvinyl alcohol, polyvinyl acetate, polyvinylpyrrolidone, polymethacrylic acid derivatives, cellulose derivatives such as ethyl cellulose, hydroxypropylmethylcellulose, triglycerides or waxes, lipids, fatty acids or fatty acid derivatives.

15. The modified release pharmaceutical composition according to claim 1, wherein the composition provides an in-vivo plasma profile for carbidopa when administered in a fasted state:
a mean of Cₘₐₓ more than about 40 ng/mL,
a mean of AUC_{0-∞} more than about 150 ng*hr/mL; or
a mean of Tₘₐₓ at least about 3 hours.

16. The modified release pharmaceutical composition according to claim 1, wherein the composition provides an in-vivo plasma profile for levodopa when administered in a fasted state:
a mean of Cₘₐₓ more than about 600 ng/mL,
a mean of AUC_{0-∞} more than about 2000 ng*hr/mL; or
a mean of Tₘₐₓ at least about 3 hours.

17. A process for preparing the modified release pharmaceutical composition according to claim 1 comprising
preparing drug pellets of carbidopa and levodopa,
coating one part of the drug pellets with one or more pH independent polymers, coating the remaining part of the drug pellets with one or more pH dependent polymers,
preparing core tablets of the organic acidic excipient, coating the tablets of the organic acidic excipient with controlled release polymers; and
encapsulating coated drug pellets of step (ii) and step (iii) and coated tablets of step (v).

18. The process according to claim 17, wherein the pellets are prepared by extrusion spheronization, solution and suspension layering, spray drying or spray congealing technique.

19. The process according to claim 17, wherein the tablets are prepared by dry granulation, wet granulation, direct compression or melt granulation.

20. A method of treating parkinson's disease comprising administering to a human patient in need thereof the modified release pharmaceutical composition according to claim 1.
